(19) United States
(12) Patent Application Publication
(10) Pub. No.: US 2012/0015021 A1
(43) Pub. Date: Jan. 19, 2012

(54) ANTI-APPETITE ADHESIVE COMPOSITIONS

(75) Inventors: Boaz Mizrahi, Brookline, MA (US); Benny Brama, Raanana (IL); Abraham J. Domb, Efrat (IL)

(73) Assignee: Axiomedic Ltd.

(21) Appl. No.: 12/835,591

(22) Filed: Jul. 13, 2010

(51) Int. Cl. A61K 36/00; A61K 36/82; A61P 3/00; A61K 36/48; A61K 36/906; A61K 36/258; A61K 9/28; A61K 36/27

(52) U.S. Cl. 424/435; 424/725; 424/434; 424/729; 424/757; 424/756; 424/728

(57) ABSTRACT

Compositions for appetite suppression and methods of making and using thereof are disclosed herein. The compositions are typically in the form of a tablet, such as a single or multi-layer sticker tablet. The compositions adhere to a buccal surface or mucosal surface in the oral cavity for at least 15 minutes, preferably for at least 30 minutes. In a preferred embodiment, the compositions contain herbal agents that are anti-appetite agents. The composition is generally effective for suppressing appetite for a time period ranging from at least 30 minutes up to eight hours following administration to the buccal or oral mucosa.

### FIELD OF THE INVENTION

This invention relates to compositions and methods for appetite suppression and overall weight management.

### BACKGROUND OF THE INVENTION

Obesity is a growing epidemic in the developed world. Over two-thirds of Americans are overweight (corresponding to a Body Mass Index greater than 25 kg/m²) and greater than one-third are obese (corresponding to a BMI greater than 30 kg/m²) according to the Centers for Disease Control and Prevention. Individuals who are obese have at least a 50 percent increased risk of premature death. In addition to the risk of death, excess body fat is associated with a variety of concomitant medical afflictions including sleep apnea, arthritis, increased disability, gout, stroke, hypertension, hypercholesterolemia, hyperlipidemia, atherosclerosis, cardiovascular disease, diabetes, cancer, bone and joint injury, metabolic syndrome, and depression.

Although obesity is a preventable disorder, the number of overweight and obese individuals is rising. The cost of treating obesity or diseases in which obesity is a contributing factor is enormous. The combined estimated cost of treating obesity-related illnesses in the United States was $147 billion in 2008.

A number of options are available to prevent weight gain and promote weight loss. Surgical modification of the gastrointestinal system has been extremely successful in helping individuals shed large amounts of weight. However, risk is inherent in any surgery, and not all patients can undergo surgical procedures due to health problems, financial constraints, and comfort level with the procedure.

The time-tested method of preventing or treating overweight individuals is modification of the balance between caloric intake and caloric expenditure, such that the number of calories burned is greater than the number of calories eaten or imbibed. Attaining the desired caloric balance can be very difficult with traditional diet modification and exercise. Accordingly, this approach has a low success rate, with 35% to 65% gaining at least some portion of the weight within a year.

A number of pharmaceuticals have been developed to alter either caloric intake or caloric expenditure. Induction of the sympathetic nervous system, generally decreased in overweight individuals, serves to increase the “flight” response, and thereby increase caloric expenditure. For example, the sympathomimetic amphetamine stimulates thermogenesis, increasing the basal metabolic rate, and stimulates lipolysis in animal models. However, activity observed in animal models has not translated to effective weight loss treatments in human clinical trials.

Caloric intake, including the perceived suppression of hunger or appetite, is mediated in the feeding and satiety center of the brain, located in the hypothalamus via a complex signaling pathway. The pathway is influenced by small molecules in the blood and catecholamine neurotransmitters such as norepinephrine, serotonin and dopamine. Optimally, the two centers balance one another so that individuals feel satiated after eating. However, a number of factors can lead to imbalance between feeding and satiety, including stress, hormonal fluctuations, and prescription medications. Administration of neurotransmitter modulators increases the availability of the catecholamines, thereby reducing the feeling of hunger. By assuaging one's appetite, less food is eaten, and overall caloric intake is lowered. Examples of such substances include phenylpropanolamine, phentermine, and the amphetamines.

However, as with all pharmaceuticals, various unwanted effects side effects are associated with treatment. Common untoward effects include excessive perspiration, dry mouth, tachycardia, hypertension, and insomnia. Furthermore, some individuals will “plateau” in their weight loss program, or become unresponsive to further treatment.

Thus, there exists a need for a safe, effective composition that promotes weight loss and maintenance by decreasing appetite.

There exists a further need for a composition form that is convenient and releases anti-appetite agents over an extended period of time for long-term appetite suppression, resulting in subsequent decreased caloric intake.

### SUMMARY OF THE INVENTION

Compositions for appetite suppression and weight maintenance, and methods of making and using thereof are disclosed. The compositions contain one or more herbal appetite suppressants and are typically in the form of a bioadhesive sticker tablet. In some embodiments, the composition is in the form of single layer, double layer, or multi-layer sticker tablet. The compositions adhere to a buccal surface or mucosal surface in the oral cavity with full dissolution after at least about 15 minutes, preferably thirty minutes, most preferably at least about four hours. The dissolution of the composition results releases anti-appetite agents in a controlled manner between meals. The compositions optionally contain a non-lipid lubricant, a flavoring agent, and/or a buffering agent. In a preferred embodiment, the composition contains one or more bioadhesive materials, one or more anti-appetite agents, one or more buffering agents, one or more flavoring and/or coloring agents, and one non-lipid lubricant. In other embodiments, the composition can further include lipid lubricants.

The compositions can be administered to individuals who are overweight, obese, dieting, or over-eating. The methods for inducing appetite suppression require placing the composition on the oral mucosa, preferably on the palate or the cheek. The composition will adhere to the mucosal surface, and dissolve, for at least about 15-30 minutes, preferably about two to four hours. The composition is generally effective for decreasing the hunger an individual feels over a time period ranging from at least about 30 minutes up to about eight hours.

### DETAILED DESCRIPTION OF THE INVENTION

### I. Definitions

“Adhesive,” as generally used herein, refers to any substance, organic or inorganic, natural or synthetic, that is capable of surface attachment to the intended application site.

“Anti-appetite agent,” as generally used herein, refers to a substance that effects decreases food intake (appetite suppressant), increases fat oxidation, increases metabolism, stimulates thermogenesis, or otherwise promotes weight loss.

“Appetite-suppressing,” as generally used herein, refers to a statistically significant and detectable or measurable reduction in food intake (over a time period of at least about 24 hours) when food is available on an ad libitum or equivalent schedule.

“Bioadhesive”, as generally used herein, refers to a material which attaches, and preferably strongly attaches, to mucosal tissue upon hydration. The material must be capable of remaining adhered to the tissue in moist or wet in vivo environments. The compositions described herein are “self-bioadhesive” in that they attach to the site of interest without the need to reinforce attachment by way of another adhesive material. The strength of adherence can be measured by standard tests for measuring the force, e.g. in dynes per square centimeter, as disclosed in U.S. Pat. No. 4,615,697 to Robinson.

“Biocompatible”, as generally used herein, means not having toxic or injurious effects on biological function in humans.

“Herbal agents” and “herbal active agents” are used interchangeably herein and generally refer to a plant or plant part used for its therapeutic properties. The herbal agents described herein are effective as a tool for weight management and appetite suppression. The herbal agent may be a plant extract, tea, oil (e.g. essential oil), tincture, etc.

“Essential oil” and “volatile oil” are used interchangeably herein and generally refer to a liquid with a high vapor pressure or low boiling point, usually having the characteristic odor or flavor of the plant from which it is obtained. Volatile oils evaporate at standard temperatures and pressures.

“Lipid”, as generally used herein, refers to any fat-soluble (hydrophobic) naturally-occurring or man-made molecule.

“Plant extract”, as generally used herein, refers to compounds and materials obtained from plants.

“Lubricant,” as generally used herein, refers to a substance which reduces friction. Lubricants can be synthetic or natural substances.

“Residence time,” as generally used herein, refers to the duration of time the tablet adheres to a mucosal surface without complete dissolution. In embodiments wherein the composition is made of non-dissolvable bioadhesive materials such as those in nicotine chewing gum, “residence time” refers to the time until the non-dissolvable bioadhesive materials are removed from the site of application.

“Tincture”, as generally used herein, refers to a plant extract prepared by steeping or soaking one or more plant materials in an alcohol or alcohol-water solvent.

### II. Compositions

The compositions contain one or more bioadhesive materials and one or more herbal appetite suppression agents. The compositions have two functions: release over a prolonged period of appetite suppressant, and retention in the oral cavity on the mucosa for a prolonged period. The prolonged retention at the site increases the amount of rapid uptake into the blood stream and may have a psychological effect since it is present in the oral cavity where food would normally be present. The prolonged release also increases the likelihood the individual will feel more sated for a longer period of time after ingestion.

The compositions suppress appetite in the patient for a prolonged period. Typically, the compositions decrease hunger for at least 30 minutes following administration, preferably for at least one, two, three or four hours following administration, up to eight hours following administration.

A. Bioadhesive Materials

The compositions contain one or more bioadhesive materials, and require sufficient retention times in the oral or mucosal cavity to provide extended appetite suppressing effects before complete dissolution. To meet this requirement, the bioadhesive compositions preferably include an anionic polymer. The anionic polymer should have a large number of hydrophilic, polar groups, such as the carboxylic acids pendant on carboxyvinyl polymers. In preferred embodiments, carboxylic acid content should be between about 50% to about 80%, more preferably between about 56% and about 68%, when assayed by standard techniques.

Suitable bioadhesive materials include, but are not limited to, carboxylic acid-containing polymers such as copolymers of acrylic or methacrylic acid; esterified polyacrylic acid polymers, such as polyacrylic acid polymers lightly crosslinked with a polyalkenyl polyethers (commercially available from B.F. Goodrich, Cincinnati, Ohio, under the trademarks CARBOPOL® 934, 934P, 974, 940 and 941); polyvinyl pyrrolidone (“PVP”); maleic acid copolymers; polysaccharides such as karaya gum, tragacanth gum, xanthan gum, jaraya gum, pectin, guar gum, locust bean gum, psyllium seed gum, alginates, hydrocolloid gels prepared from polysaccharides extracted from Fronia elephantum, Sapindus trifoliatus, Kunjac, and the cashew tree; celluloses such as carboxymethyl cellulose, hydroxypropyl cellulose (HPC, Klucel®), and mixtures thereof, and mixtures of sulfated sucrose and aluminum hydroxide, and other substances capable of forming a solid colloid that can adhere to tissue, used alone or in combination with other suitable carriers.

A preferred bioadhesive is CARBOPOL® 934 or an equivalent, and is a water-soluble polymer of acrylic acid crosslinked with about 1% of a polyallyl ether of sucrose having an average of about 5.8 allyl groups for each sucrose molecule.

In preferred embodiments, the bioadhesive material is esterified polyacrylic acid polymers (CARBOPOL® 934, 934P, 974, 940 and 941), cellulose and cellulose derivatives (carboxymethyl cellulose, hydroxypropyl cellulose), and combinations thereof.

In some embodiments, the bioadhesive materials contain at least one crosslinked polyacrylic acid, such as CARBOPOL® 934 or 971. In a preferred embodiment, the bioadhesive material is a mixture of crosslinked polyacrylic acid, i.e. CARBOPOL® 934, polyvinyl pyrrolidine (PVP), and hydroxypropyl cellulose (HPC).

The concentration of the bioadhesive materials in the composition ranges from 5% to 50% by weight of the tablet, more preferably from about 10-40% by weight of the tablet, more preferably from 15-35% by weight of the tablet, most preferable from 25-35% by weight of the tablet.

In some embodiments, the composition is a multi-layer tablet, preferably a double layer tablet, containing a bioadhesive layer. The bioadhesive materials in the bioadhesive layer are present in 10% to 100% by weight of the bioadhesive layer, preferably from about 10% to 50% by weight of the bioadhesive layer, more preferably from 20-50% by weight of the tablet, more preferably from about 30-50%, most preferably about 45% by weight of the bioadhesive layer.

In some embodiments, the tablets contain multiple layers. In embodiments wherein the tablet contains two layers, the bioadhesive layer adheres to the oral cavity. The outer layer, facing the interior of the mouth, serves as a delivery layer for the herbal agent and/or flavoring. In another embodiment, the tablet has more than two layers, wherein the bioadhesive layer adheres to the oral cavity and other layers contain active agents. In multiple layer embodiments, bioadhesive materials may be included in any active agent delivery layer to allow adhesion to the bioadhesive layer. The concentration of the bioadhesive materials in the delivery layer of a multilayer tablet is between 1% to 50% by weight of the tablet, more preferably from 10% to 40% by weight of the tablet, more preferably from 20% to 40% by weight of the tablet, more preferably from 30% to 40% by weight of the tablet, most preferably in about 33% by weight of the tablet.

B. Active Agents

Natural extracts obtainable from plants of the Apocynaceae family (also known as the Asclepiadaceae family), particularly the Hoodia genus (formerly the Hoodia and Trichocaulon genera) have been shown to have an appetite suppressant activity. U.S. Pat. No. 6,376,657 to Van Heerden, et al. discloses that these plants contain anti-appetite steroidal glycosides and processes to extract steroidal glycosides from Hoodia plants.

U.S. Patent Application No. 2006/0105068 to Fleischner discloses diet compositions for weight control including effective amounts of hoodia gordonii cactus, alone or together with chromium, vanadium amino acid chelate, glucomannan, sodium carboxymethylcellulose, citrus naringinine, green tea, cocoa extract, glucosamine HCl, ma huang, 3-acetyl-7-oxo-dehydroepiandrosterone, and coleus forskohlii. U.S. Patent Application No. 2008/0138447 to Riggins, et al. discloses compositions including Guarana extract, Hoodia, Cha de bugre, Ginseng, Citrus Auratium, Magnolia bark extract, and black pepper, for suppressing appetite. These compositions prevent an individual from eating excessively during a meal, but do not prevent the craving for food before meals or between meals.

U.S. Patent Application No. 2007/0196436 to Abrahamse, et al. discloses edible aqueous dispersions of one or more steroidal glycosides. U.S Patent Application No. 2007/0104805 to Udel, discloses soft gelatin capsules that contain a combination of an extract from the genus Trichocaulon, including Trichocaulon officinale or Trichocaulon piliferum, or from the genus Hoodia, including Hoodia currorii, Hoodia gordonii or Hoodia lugardii and an extract from the genus Pinus containing pinolenic acid. The dispersion or gelatin capsule can be taken between meals, but does not have a lasting effect with regard to appetite suppression. In addition, both forms will undergo first pass metabolism, which may compromises the effectiveness of treatment and means that higher dosages must be administered.

In preferred embodiments, the compositions contain one or more herbal anti-appetite agents. In one embodiment, each layer of a multi-layer tablet contains one herbal anti-appetite agent. In other embodiments, each layer of a multi-layer tablet contains multiple herbal anti-appetite agents.

In the preferred embodiment, the appetite suppressant tablet includes an extract of a plant of the genus Hoodia or Trichocaulon, in particular Hoodia gordonii. In one or more embodiments, Hoodia is extracted from a cactus indigenous to South Africa. Hoodia extract includes one or more saponin compositions that interact directly with the brain's appetite system to suppress a user's food intake cravings. Hoodia extract is available from AHD International, Inc. of Atlanta, Ga.

Alternatively, Hoodia or Trichocaulon can be isolated by extraction from plant sap followed by spray-drying or solvent extraction. The extract can be prepared from plant material such as the stems and roots of plants of the genus Hoodia or the genus Trichocaulon. The plant extract is generally obtained from one of the species: Trichocaulon piliferum; Trichocaulon officinale; Hoodia currorii; Hoodia gordonii; and Hoodia lugardii.

Extracts from Hoodia or Trichocaulon provide steroidal glycosides, which cause the brain to think that the stomach is “full,” and thereby act as appetite suppressants. One such steroidal glycoside of importance is known as P57 or P57AS3. The identification and isolation of P57 and Hoodia and Trichocaulon extracts are found in U.S. Pat. No. 6,376,657 to Van Heerden.

In a preferred embodiment, the anti-appetite agent is green tea. Green tea is known to accelerate calorie burning via increased thermogenesis. Green tea contains a number of polyphenolic compounds. The catechin epigallocatechin gallate (EGCG) is the most abundant with greater than 50% of total tea catechins, and is the active agent. The other main catechins are epicatechin (EC), epicatechin gallate (ECG), and epigallocatechin (EGC).

In another embodiment, the anti-appetite agent is bitter orange. Bitter orange (citrus aurantium) is fat burner that increases the metabolic rate. The active agent in bitter orange is synephrine, which stimulates the adrenal gland to affect fat burning and appetite suppression.

In another embodiment, the anti-appetite agent is yerba mate. Yerba mate oxidizes body fat, enhancing the rate at which fat is broken down. The active ingredient in yerba mate is mateine, which enhances energy levels, and suppresses an individual's appetite while avoiding jitteriness, nervousness or stomach aches.

In another embodiment, the anti-appetite agent is joboba. Jojoba seed (Simmondsia chinensis), called Simmondsin, is a natural appetite suppressant.

In another embodiment, the anti-appetite agent is a guggul lipid. Gum Guggul (Commiphora mukul), also known as Guggul, Indian Bedellium, and Guggulow is a gum resin from the mukul myrrh tree. Guggul lowers cholesterol levels and protects against hardening of the arteries. The active agents in Guggul include phytosterols, gugulipids, and guggulsterones. Guggul is also a weight loss agent.

In another embodiment, the anti-appetite agent is ephedra (Ephedra sinica), also known as Ma Huang. Ephedra contains two active agents that are alkaloids, ephedrine and pseudoephedrine. Ephedra suppresses the appetite and stimulates the thyroid gland to upregulate metabolism and thermogenesis.

In another embodiment, the anti-appetite agent is garcinia cambodgia (also known as citrin or gambooge). Garcina cambodgia is rich in hydroxycitric acid (HCA), which promotes weight loss by blocking the conversion of sugary foods and starches into fats, raises levels of the brain chemical serotonin, and suppresses appetite.

In another embodiment, the anti-appetite agent is fenugreek (Trigonella foenumgraecum). Fenugreek regulates blood sugar regulation and/or glucose metabolism.

In a preferred embodiment, the anti-appetite agent is Panax ginseng (also known as ginseng, Korean ginseng, schinsent, or ninjin). Ginseng is an adaptogen that lowers cholesterol, balances the metabolism, increases energy levels, and stimulates the immune system.

In another embodiment, the anti-appetite agent is extract of lotus root, lotus leaf, or lotus seed that provides asparaginic acid and vitamin B12.

In another embodiment, the biologically active substance may include substances that support the subject's weight management control. Substances that support weight management control may be selected from, but not limited to, almond, Aloe vera, alpha lipoic acid, aminogen, ammonium glycyrrhizate, amylum fruit extract, astaxanthin, bean pod, biotin, Piper nigrum L (black pepper) extract, Piper longum L (long pepper) extract, black tea extract, bladderwrack (kelp), blue-green algae, broccoli, Indian Fig Opuntia cactus, caffeine, caralluma, carob, cassia seed extract, cayenne, calcium, calcium phosphate, cedarwood oil, cetyl alcohol, chitosan, Cissus quadrangularis extract (stem & leaves), citrus lime oil, citrus orange oil, cocoa, CoEnzyme Q10, coix seed, cola nut, Coleus forsholii extract, cujquat (fruit), Combreturn micranthum (leaf) extract, cyclometicone, dandelion root, dehydroepiandrosterone (DHEA), 7-keto-DHEA, dill weed, diacylglycerol, dimethicone, disodium succinate, DL-phenylalanine, fibs citri auranti (blossoms), folic acid, Garcinia cambogia extract, geranium oil, ginger root, American root, fish oil, panax extract, grapefruit oil, guaiacwood oil, guarana extract, Gymnema sylvestre, glucomannan, 5-hydroxy-tryptophan (5-HTP), inulin, kahkow fruit extract, lavendin oil, lecithin, hydroxylated, lemon grass, licorice, linoleic acid, L-camitine, L-glutamine, L-methionine, L-tyrosine, Lespedeza capitata extract, Litsea cubeba fruit oil, magnolia, methyl paraben, milk protein isolate, mulberry (leaf), nettle leaf, niacin/niacinamide, Oolong tea extract (Camellia sinensis), pantothenic acid, papaya leaf, PEG-12/PEG-100, phaseolamin, phellodendron, picamilon HCl, pine leaf oil, potassium citrate, potassium iodide (iodine), potassium phosphate, Poria cocas (Fu Ling), propylparaben, pyruvate, quercetin, red clover blossom, Rhodiola rosea extract, rhubarb root (Da Huang), Rooibos tea extract (leaf & stem), rosemary leaf oil, sesame oil (Sesamum indicum), senna (leaf), Caralluma fimbriata, soy lecithin, soy protein isolate, Spanish sage oil, stevia leaf, sunflower oil, tangerine oil, tarragon extract tiratricol, Ulva lactuca extract, Arctostaphylos uva ursi leaf, vinpocetine, vitamin A, vitamin B1/B2, vitamin B6/B12, vitamin C (ascorbic acid), vitamin D3, vitamin E (d-alpha-tocopherol), water plantain rhizome (stem), whey powder, whey protein isolate, white willow bark extract, white pepper, Withania somnifera extract, Wu-Long tea, Yellowdock root, zinc oxide, and combinations thereof.

In a preferred embodiment, the herbal agent is hoodia gordonii, alfalfa C-106, ginger, or a combination thereof.

In one embodiment, the one or more herbal agent is present in a single layer tablet in an amount between about 1% and 50% by weight of the tablet, more preferably between 10% and 30% by weight of the tablet, and most preferably between 20% and 30% by weight of the tablet. The concentration of any one particular herbal agent is present in an amount between about 1% and 50% by weight of the tablet, more preferably between 10% and 30% by weight of the tablet, and most preferably between 20% and 30% by weight of the tablet.

In one embodiment, the one or more herbal agent is present in a multi-layer tablet in an amount between about 1% and 50% by weight of the tablet, more preferably between 1% and 20% by weight of the tablet, and most preferably between 5% and 12% by weight of the tablet. The concentration of any one particular herbal agent is present in an amount between about 1% and 50% by weight of the tablet, more preferably between 1% and 20% by weight of the tablet, and most preferably between 3% and 12% by weight of the tablet.

C. Carriers, Additives and other Excipients

### Buffering Compounds

Compositions are prepared using a pharmaceutically acceptable carrier composed of materials that are considered safe and effective and may be administered to an individual without causing undesirable biological side effects or unwanted interactions. The term “carrier” includes, but is not limited, to diluents, binders, stabilizers, flavoring agents, pigments, humectants, disintegrators, and fillers.

Suitable humectants include, but are not limited to, edible polyhydric alcohols, such as glycerin, sorbitol, xylitol, butylene glycol, polyethylene glycol, and propylene glycol. In one embodiment, the humectant is sorbitol and/or glycerin. The humectant may also act as a plasticizer to provide a flexible sticker, which is comfortable to the user when placed in his/her mouth. The concentration of the humectant is from about 1% to about 20% by weight of the composition, preferably from about 1% to about % by weight of the composition.

Preferably the composition contains one or more flavoring agents, such as natural or artificial sweeteners. This is particularly preferred if the anti-appetite agent is flavorless or has an unpleasant taste. Suitable flavoring agents include, but are limited to, oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and combinations thereof. The concentration of the flavoring agent is from about 0.001% to about 1% by weight of the composition.

Natural or artificial sweeteners include, but are not limited to, sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and combinations thereof. Preferably the sweetener is a non-cariogenic sugar alcohol. The concentration of the sweetener is from about 0.1% to about 5% by weight of the composition, preferably about 4% by weight of the composition, although concentrations as great as 40% may be used.

Flavoring agents (including sweeteners) are present in the tablet at 0.1% to 5% by weight, depending on the specific flavor and desired attributes. If plant-based or herb-based sialogogic agents are incorporated into the compositions, these may be included at 0.25% to 20% of the composition by weight. Artificial sweeteners are used according to taste, and generally the composition contains at least 0.1% (wt/wt) of an artificial sweetener.

Tabletting materials such as binders, fillers, and flow aids typically accounts for about 90% of the mass, and comprises mostly sugar alcohols, but includes flow aids, lubricants, flavors and excipients. Suitable binders include sorbitol, lactose, urea, sucrose stearate, starch, maltodextrin, corn syrup solids, sodium citrate, sodium sulfate, sodium chloride, sucrose, and dextrates. The composition contains at least 0.1% (wt/wt) of a binder.

Desensitizing agents may also be added, such as strontium nitrate and potassium nitrate, to reduce heat or cold sensitivity.

Antimicrobial agents such as cetylpyridinium chloride and domiphen bromide may be added to reduce bacterial levels in the oral cavity.

Breath freshening ingredients such as chlorophyll may be added and pharmaceutical agents such as antibiotics may also be included in the compositions.

Non-limiting examples of some other components that may be included in a delivery composition include one or more of the following: penetration enhancers, stabilizers for the xerostomia therapeutic, preservatives such as antioxidants, butylated hydroxytoluene, antifungals, and antibacterials.

The composition typically includes one or more buffering compounds in an effective amount to maintain a neutral pH in the oral cavity for at least 30 minutes following administration. As the composition dissolves in the oral cavity, it generates a buffered solution in the oral cavity which, by maintaining a relatively neutral pH, can help to diminish the formation of caries and renders the oral cavity less prone to infection.

Preferred buffering compounds include disodium hydrogen phosphate, calcium chloride, citric acid, sodium citrate or potassium citrate, sodium acetate, ethanolamine, or a combination thereof. Other suitable buffering compounds include acids, such as fumaric acid, tartaric acid, malic acid, adipic acid, and other edible acids or their pharmaceutically acceptable salts can be used. Sodium carbonate and sodium bicarbonate are the preferred carbonate salts. However carbonates and bicarbonates of potassium, sodium, ammonium, magnesium, and calcium can also be used. The composition may contain from about 1% to about 10% by weight of the buffering compound.

The composition optionally includes one or more excipients which increase the time period during which the buffered solution remains in the oral cavity, as compared to the same composition in the absence of such excipients. Exemplary excipients include, but are not limited to, glycerin, polymers including, but not limited to, natural gums including Xanthan (e.g., 0.5 to 15% by weight), cellulose based materials such as methylcellulose, carboxymethylcellulose, hydroxypropylmethyl cellulose, hydroxyethyl cellulose (e.g., 0.2% to 40% by weight), acrylic acids including crosslinked and non-cross linked acrylic polymers, (e.g., 0.2% to 50% by weight), polyethylene glycols (e.g., 0.2% to 2% by weight), dextran (e.g., 0.05% to 0.5% by weight), gelatin (e.g., 0.2% to 5% by weight), polyvinyl alcohol (e.g., 0.1% to 5% by weight), polysorbate 80 (e.g., 0.2% to 2% by weight), and polyvinyl pyrrolidine (e.g., 0.1% to 4% by weight).

D. Lubricants

In some embodiments, the compositions include non-lipid lubricants. Non-lipid lubricants should be food-grade materials. Suitable non-lipid lubricants include, but are not limited to, hydrogels, such as CARBOPOL® (Lubrizol Advanced Materials, Inc.), water soluble polymers such as polyethylene glycol (molecular weight from 400-1,000,000), glycerol, polypropylene glycol, polyvinyl alcohol, polyvinyl pyrrolidone (“PVP”) (e.g. PVP K-30 and/or PVP K-90), sodium benzoate, leucine, magnesium stearate, sodium lauryl sulfate, and sodium lauryl sulfoacetate. The concentration of the non-lipid lubricant is from about 3% (wt/wt) to about 80% (wt/wt). In a preferred embodiment, the non-lipid lubricant is present from about 40% (wt/wt) to about 70% (wt/wt). In a more preferred embodiment, the non-lipid lubricant is present in about 66% (wt/wt). In embodiments wherein the composition is in the form of a double layer tablet, the non-lipid lubricant is present in the bioadhesive layer from about 10% (wt/wt) to about 50% (wt/wt), more preferably from about 20% (wt/wt) to about 40% (wt/wt), and most preferably in about 37% (wt/wt).

In some embodiments, the compositions contain one or more lipids. Lipids include fatty-acids and their derivatives (including tri-, di-, and monoglycerides and phospholipids), fatty alcohols and their derivatives, as well as other fat-soluble sterol-containing metabolites such as cholesterol. In other embodiments, the lipid lubricant is a triglyceride, including, but not limited to, tricaprin, trilaurin, triacetin, trimyristin, and triolein. In other embodiments, the lipid lubricant is a phospholipid, including, but are not limited to, phosphoglycerides (e.g. phosphatidyl serine, phosphatidyl inositol, phosphatidyl ethanolamine, and phosphatidyl choline) and sphingomyelin.

Preferred lipids are generally those that melt at or around body temperature so that they are solid at room temperature, but semiliquid or liquid at body temperature. Examples of preferred lipids include, but are not limited to, tricaprin, ethyl stearate, short chain waxes, and partially hydrogenated plant oils, such as corn oil. Additional preferred lipids include mixtures or pure mono-, di-, and triglycerides, semisolid phospholipids and hydrophobic short chain polymers, such as polycaprolactone.

The compositions contain an effective amount of the lipid to reduce friction and lubricate the mouth following administration of the composition. The concentration of the lipid in the composition is at least about 5% by weight of the composition, and is no greater than about 50% by weight. Preferably the composition contains at least about 10% by weight of lipid, and more preferably the concentration of lipid in the composition ranges from about 10% to about 30% (wt/wt).

E. Forms of the Composition

The compositions can be in form of a single layer, double layer, or multilayer tablet, which can be prepared using conventional methods known in the art, such as by compression tableting. In one embodiment, the tablet is a single layer table. In another embodiment, the tablet is a multi-layer tablet. In a preferred embodiment, the composition is a double layer tablet.

The total mass of the sticker tablet generally ranges from 50 mg to 1000 mg, depending on particular consumer preferences and desired performance attributes such as composition residence time in the mouth. The preferred mass ranges from 200 mg to 500 mg.

The tablets can be of any suitable size for placement in a patient's mouth. In one embodiment, the surface area of the tablet is from about 0.4 cm² to about 3 cm², preferably from about 0.5 cm² to 1.8 cm², more preferably from 0.5 cm² to 1.2 cm². For example, a tablet having a diameter of 15 mm, will have a surface area of approximately 1.8 cm². In the most preferred embodiment, the tablet has a suitable geometry for placement on the desired surface in the mouth. For example, for placement on the palate, the table preferably contains a convex surface designed to be placed adjacent to and adhere to the palate. In a double layer tablet, this side corresponds with the outer surface of the bioadhesive layer of the tablet. Tablets are typically round or oval, with a diameter up to 3 cm.

In one embodiment, the single layer bioadhesive sticker tablet contains 10-30% CARBOPOL® 934 or CARBOPOL® 971, 5-15% hydroxypropyl cellulose (Klucel HF), 5-20% polyvinyl pyrrolidine, 5-20% xylitol/carmine premix, 0.5-1% magnesium stearate, 1-5% lemonade flavoring (#02-FF992), 1-3% mint oil, 1-50% hoodia gordonii, 1-50% green tea, 1-50% alfalfa C-106, and 1-50% ginger or ginseng (ginseng is preferred) by weight of the tablet.

In a preferred embodiment, the single layer bioadhesive sticker tablet contains about 25% CARBOPOL® 934 or CARBOPOL® 971, about 8.0% hydroxypropyl cellulose (Klucel HF), about 16% polyvinyl pyrrolidine, about 16% xylitol/carmine premix, about 1% magnesium stearate, about 4% lemonade flavoring (#02-FF992), about 2% mint oil, about 16% hoodia gordonii, about 16% green tea or alfalfa C-106, and about 6% ginger or ginseng (ginseng is preferred) by weight of the tablet.

In one embodiment, a double layer bioadhesive sticker tablet contains a bioadhesive layer and an outer delivery layer that does not adhere to the mucosa. The bioadhesive later contains 1-50% CARBOPOL® 934 or CARBOPOL® 971, 1-30% hydroxypropyl cellulose (Klucel HF), 20-50% polyvinyl pyrrolidine, 1-20% xylitol/carmine pre-mix, and 1-5% magnesium stearate by weight of the tablet. In a preferred embodiment, the bioadhesive layer contains about 30% CARBOPOL® 934 or CARBOPOL® 971, 15% hydroxypropyl cellulose (Klucel HF), 37% polyvinyl pyrrolidine, 13% xylitol/carmine pre-mix, and 2% magnesium stearate by weight of the tablet.

In one embodiment, the outer delivery layer contains 1-10% calcium carbonate, 1-10% sodium chloride, 10-50% polyvinyl pyrrolidine, 1-5% silicon dioxide, 5-50% CARBOPOL® 934 or CARBOPOL® 971, 10-30% xylitol/carmine pre-mix, 1-10% lemonade flavoring (#02-FF992), 1-5% mint oil, 1-50% hoodia gordonii, 1-10% green tea, 1-10% or alfalfa C-106, and 1-10% ginger or ginseng. In a preferred embodiment, the outer delivery layer contains about 4.5% calcium carbonate, about 3% sodium chloride, about 21% polyvinyl pyrrolidine, about 1.5% silicon dioxide, about 15% CARBOPOL® 934 or CARBOPOL® 971, about 21% xylitol/carmine pre-mix, about 6% lemonade flavoring (#02-FF992), about 1.5% mint oil, about 15% hoodia gordonii, about 6% green tea or alfalfa C-106, and about 6% ginger or ginseng.

### III. Method of Making the Compositions

A. Compression Molding

Compression molding can be used to prepare single layer, dual layer, or multilayer sticker tablets. The simplest method for preparing the sticker tablets is by compression molding using a single or multi-punch press machine. The powder is loaded in the punch having a diameter ranging from about 4 to about 15 mm and a thickness of about 0.5 mm to about 2.5 mm. The thickness is defined by the amount of powder added, usually between about 50 mg and 250 mg. The powder is compressed to form a single layer sticker tablet.

Double layer sticker tablets are prepared using the double compression technique. The inert powder is first added to the punch to cover the surface. The formulation powder is added on top and compression is applied to produce a sticker tablet where one side is bioadhesive and the other is not. The non-bioadhesive side also tends to be less water-permeable than the bioadhesive side. Alternatively, one powder is added to the punch and compressed to form a thin tablet. The second powder is then added and compressed to form a uniform bilayer tablet.

B. Spray Coating

Double layer sticker tablets can also be prepared by spray coating. In the spray coating method, the coating is applied by spraying an alcoholic solution or fine dispersion of a hydrophobic coating material onto one side of the sticker tablet. The spray coating can be applied using an automated machine where the tablets are placed onto a running sheet which is exposed to spray nozzles to spray coat the tablets. Typical hydrophobic powders suitable for this coating include: fatty acids and salts such as Mg- or Ca-stearate, triglycerides and fatty acid esters, ethyl cellulose, methyl methacrylate-methacrylic acid copolymers (EUDRAGIT®), and other pharmaceutically acceptable hydrophobic components.

C. Solvent Casting

Another way of preparing thin single layer sticker tablets is by casting a concentrated suspension in ethanol of all tablet ingredients onto a flat surface where, after solvent evaporation, a thin sheet is obtained. The sheet is then cut into films of the desired size and shape using a cutting mold.

Double layer films can be prepared by applying the coating as a spray on top of the sheet loaded with the active agents. Other industrial methods can be used, such as forming the sheet on an edible hydrophobic sheet such as rice paper and cutting the sheets into the desired size.

D. Controlled Release Compositions

In some embodiments, the tablet or film is designed for controlled release of the anti-appetite agent.

In one embodiment, an anti-appetite agent is absorbed in or onto a polymeric component or is encapsulated into microcapsules that control the release of the agent when embedded in the tablet. For example, a solution (e.g. ethanolic solution) of a herbal extract may be absorbed in a polymer matrix, such as crosslinked polyacrylic acid (e.g. CARBOPOL®), hydroxyl propyl cellulose (HPC), ethyl cellulose or EUDRAGIT® powders, and added to the tablet mixture, preferably on the outer layer.

Encapsulation of anti-appetite agents in matrix type or capsule type particles can be done via standard methods used in the pharmaceutical industry. Encapsulating materials include, but are not limited to, ethyl cellulose, copolymers of methacrylic acid and methyl methacrylate, gelatin, alginates, gum polysaccharides, polycyanoacrylate, etc.

Encapsulation processes are selected or designed taking into consideration the heat sensitivity and the low melting or boiling point and/or the sublimation temperature of the sialogogic agents of interest.

E. Inclusion of Biological Agents in Tablet or Film

When biological agents, such as probiotic agents and enzymes, are included in the tablet or film, they are typically incorporated as a dry powder. Prior to the addition of the biological agents to the other materials for the formation of the tablet or film, the biological agents may be stabilized with a mixture of amino acids, salts, and acidic and basic small molecules, may be encapsulated in a polymeric carrier or may be absorbed in a pharmaceutically acceptable excipient or additive, such as polysaccharides or acrylate-based polymers.

### IV. Methods of Administering the Compositions

The compositions are intended for appetite suppression, weight loss, a method to increase glucose metabolism, a method to reduced body fat, and to help the individual maintain the weight loss.

The sticker tablet is applied to the oral mucosa. In preferred embodiments the compositions can be administered between meals. Preferably the tablet is placed on the palate of the mouth cavity; however it may be placed in any suitable mucosal tissue inside the mouth, such as on the cheek.

The sticker tablet is preferably administered from about once a day to four times per day, more preferably from about once per day to about twice per day. In some treatments, the patient will rest for a suitable period of time between applications of the tablet or film to allow natural activity of the mouth organs. Additionally or alternatively, in the preferred embodiment, the tablet should be attached in different locations in the patient's mouth for consecutive treatments to minimize local mucosal irritation and provide relief to the immediately prior adhesion site. For example, one tablet may be placed at a first location, such as the palate; for the second treatment, the tablet may be placed in a different location, such as on the buccal mucosa, and for a subsequent treatment, the tablet may be placed at the first location (e.g. the palate) or a location different from both the first and second placement locations.

In some embodiments, the bioadhesive tablet adheres to the buccal or mucosal surface, such as on the palate or cheek, with a residence time of at least 15 minutes following administration. In other embodiments, the residence time of the bioadhesive tablet is from about 15 minutes to about 12 hours, more preferably from about 15 minutes to eight hours. Typically the individual determines the length of time of residence by feeling the presence of the tablet.

In one embodiment, residence time is determined by complete dissolution of the tablet. In these cases, residence time is at least equal to dissolution time. In another embodiment, non-dissolvable portions of the tablet are physically removed by a medical professional or the individual user. In these embodiments, residence time can be greater than dissolution time.

In preferred embodiments, the composition does not fully dissolve for at least 15 minutes following administration to the buccal or oral mucosa, more preferably for at least 30 minutes following administration following administration, and most preferably for one, two, three or up to eight hours following administration.

In some embodiments, the layers of a multi-layer tablet will dissolve concurrently following administration to a buccal or mucosal cavity. In other embodiments, layers of a multi-layer tablet dissolve with different dissolution times following administration. In yet other embodiments, one or more layers of the tablet dissolve after administration, but other layers, the mucoadhesive layer for example, does not dissolve.

The compositions described herein offer appetite-suppressing and general weight management effects. Upon placement of the composition in the buccal or mucosal cavity, the composition begins to dissolve, providing the individual with appetite suppressing effects for at least about 15 minutes following administration, up to 8 hours. More preferably, appetite is suppressed for from about 30 minutes to eight hours following adherence of the tablet or film.

In one or more embodiments, the bioadhesive appetite suppressant tablet at least partially controls sugar craving by reducing the ability of the user's taste buds to sense sweet-tasting food products for a period of time, such as at least 15 minutes, more preferably from about 30 minutes to eight hours. One or more of the active ingredients of the appetite suppressant tablet results in mood elevation and/or stimulates the feeling of satiety. The appetite suppressant tablets of one or more embodiments may supplement a diet plan to reduce food cravings, which typically limit the success of dieting. In one embodiment, 1-3 tablets may be consumed daily.

The present invention will be further understood by reference to the following non-limiting examples.

### EXAMPLES

### Example 1

### Preparation of Double Layer Anti-Appetite Tablets

Compositions and Methods of Manufacture

The anti-appetite tablets contain two layers: layer A, which is convex and mucoadhesive, and layer B, which is flat and faces the inside of the oral cavity upon application. Layer B, the outer delivery layer, contains the anti-appetite agents. This design enables the tablet to fit snugly on the palate in the mouth.

Layer A contains Carbopol 934 (Goodrich), Klucel HF (HPC, Hercules), polyvinylpyrrolidine K90 (Fluka), xylitol/carmine pre-mix, and magnesium stearate.

Layer B contains calcium carbonate, sodium chloride, polyvinylpyrrolidine K90, silicone dioxide, Carbopol 934, xylitol, Lemonade #02-FF992 (natural), hoodia gordonii, green tea or alfalfa C-106, and ginger.

Layers A and B were prepared separately by mixing the ingredients. The tablets were fabricated using a double press machine. Layer A was added to the convex punch hole and pressed. Then, layer B was added on top of layer A and the maximum pressure was applied. The tablets were stored in a well-sealed container with dry nitrogen in a cold and dry environment.

The ingredients and amounts contained in the double layer anti-appetite agent bioadhesive tablets are provided below.

### Composition of Anti-Appetite Bioadhesive Double-Layer Sticker Tablets

### Layer A:

| | |
|---|---|
| Carbopol 934 or substitute, (Goodrich) | 30 mg |
| Klucel HF (HPC), (Hercules) | 15 mg |
| Polyvinylpyrrolidone K90, (FLUKA) | 37 mg |
| Xylitol/Carmine Pre mix | 13 mg |
| Magnesium stearate | 2 mg |
| Silicon dioxide | 3 mg |
| Total weight: | 100 mg |

### Layer B:

| | |
|---|---|
| Calcium carbonate | 15 mg |
| Sodium Chloride | 10 mg |
| Xylitol | 70 mg |
| Polyvinylpyrrolidone K-90 | 70 mg |
| MCT-70 | 30 mg |
| Citric acid | 20 mg |
| Silicone dioxide | 5 mg |
| Lemonade | 20 mg |
| Carbopol 934 or substitute, (Goodrich) | 50 mg |
| Lemonade #02-FF92 (Natural) | 20 mg |
| Mint oil | 5 mg |
| Hoodia gordonii | 50 mg |
| Green Tea or Alfalfa C-106 | 20 mg |
| Ginger | 20 mg |
| Total weight | 335 mg |

### Example 2

### Single Layer Anti-Appetite Tablets

Single layer anti-appetite tablets were prepared by combining the ingredients in the amounts provided below.

### Composition of Anti-Appetite Bioadhesive Single-Layer Sticker Tablets

| | |
|---|---|
| Carbopol 934 or substitute, (Goodrich) | 65 mg |
| Klucel HF (HPC), (Hercules) | 20 mg |
| Polyvinylpyrrolidone K90, (FLUKA) | 40 mg |
| Xylitol/Carmine Pre mix | 40 mg |
| Magnesium stearate | 2 mg |
| Lemonade #02-FF92 (Natural) | 10 mg |
| Mint oil | 5 mg |
| Hoodia gordonii | 40 mg |
| Green Tea or Alfalfa C-106 | 15 mg |
| Ginger or ginseng | 15 mg |
| Total weight | 252 mg |

Modifications and variations of the compositions and methods of making and use thereof are intended to come within the scope of the appended claims.

We claim:

1. A bioadhesive sticker tablet for appetite suppression in a patient comprising a pharmaceutically acceptable bioadhesive carrier,
wherein the tablet, upon application to a mucosal surface, adheres to the mucosal tissue for at least 15 minutes; and
wherein the tablet does not completely dissolve prior to about 15 minutes following application to a mucosal surface; and
wherein the tablet contains a therapeutically effective amount of an herbal anti-appetite agent that suppresses appetite for at least 15 minutes following administration to the oral mucosa.

2. The tablet of claim 1, wherein the tablet is in the form of a single layer tablet.

3. The tablet of claim 1, wherein the tablet is in the form of a double layer tablet.

4. The tablet of claim 3, wherein the first layer and the second layer of the double layer tablet comprise one or more bioadhesive materials.

5. The tablet of claim 1, wherein the tablet dissolves following application to the mucosal surface over a period of time from about 15 minutes to about eight hours.

6. The tablet of claim 1, wherein the anti-appetite agent is an herbal agent.

7. The tablet of claim 6, wherein the anti-appetite agent is selected from the group consisting of Hoodia gordonii, green tea, alfalfa, ginger, ginseng, and combinations thereof.

8. The tablet of claim 1, wherein the anti-appetite agent is present in a concentration of 3 to 30% by weight of the tablet.

9. The tablet of claim 8, wherein the anti-appetite agent is present in a concentration of 20 to 30% by weight of the tablet.

10. The tablet of claim 8, wherein the anti-appetite agent is present in a concentration of 3 to 12% by weight of the tablet.

11. The tablet of claim 1, further comprising a flavoring agent, a binder, or a buffering agent, or a combination thereof.

12. The tablet of claim 1, wherein a mucoadhesive is present in at least five percent by weight.

13. The tablet of claim 1, wherein the mucoadhesive is selected from the group consisting of copolymers of acrylic or methacrylic acid; esterified polyacrylic acid polymers; maleic acid copolymers; polysaccharides; hydrocolloid gels prepared from polysaccharides extracted from Fronia elephantum, Sapindus trifoliatus, Kunjac, and the cashew tree; cellulose; cellulose derivatives; or combinations thereof.

14. The tablet of claim 12, wherein the mucoadhesive comprises a polyacrylic acid polymer crosslinked with a polyalkenyl polyether and hydroxypropyl cellulose.

15. The tablet of claim 1 having a mass of from about 50 mg to about 500 mg.

16. The tablet of claim 1 having a surface area from about 0.4 cm² to about 30 cm² or has a diameter of up to three cm.

17. A method for suppressing appetite in a patient in need thereof, comprising:
administering on the buccal or oral mucosa of the mouth of the patient an adhesive tablet comprising an herbal agent that is an anti-appetite agent, effective to adhere the tablet to the mucosa for at least about 15 minutes following administration,
wherein the tablet does not completely dissolve prior to about 15 minutes following administration, and
wherein appetite is suppressed for at least about 15 minutes following administration

18. The method of claim 16, wherein the tablet is placed on the palate or the cheek.

19. The method of claim 16, wherein the tablet adheres to the buccal or oral mucosa for between about 15 minutes and about eight hours.

20. The method of claim 16, wherein the tablet dissolves following application to the mucosal surface over a period of time ranging from about 15 minutes to about eight hours.

21. The method of claim 16, wherein the mucoadhesive is selected from the group consisting of copolymers of acrylic or methacrylic acid; esterified polyacrylic acid polymers; maleic acid copolymers; polysaccharides; hydrocolloid gels prepared from polysaccharides extracted from Fronia elephantum, Sapindus trifoliatus, Kunjac, and the cashew tree; cellulose; cellulose derivatives; or combinations thereof.

22. The method of claim 23, wherein the mucoadhesive comprises a polyacrylic acid polymer crosslinked with a polyalkenyl polyether and hydroxypropyl cellulose.

23. The method of claim 17, wherein appetite is suppressed for a period of time between 15 minutes and eight hours following administration to the oral mucosa.
